(19) United States
(12) Patent Application Publication
(10) Pub. No.: US 2007/0270441 A1
(43) Pub. Date: Nov. 22, 2007

(54) Combination

(75) Inventor: Johannes Barsig, Konstanz (DE)

Correspondence Address: THE NATH LAW GROUP, 112 South West Street, Alexandria, VA, 22314, US

(73) Assignee: Altana Pharma AG, Konstanz (DE)

(21) Appl. No.: 11/826,876

(22) Filed: Jul. 19, 2007

Related U.S. Application Data
(62) Division of application No. 10/184,068, filed on Jun. 28, 2002.

(30) Foreign Application Priority Data
Nov. 9, 2001 (EP) 01 00 0607

(51) Int. Cl. A61K 31/513; A61K 31/4412; A61P 11/00; A61P 11/06; A61P 11/08; A61P 17/06; A61P 19/02; A61P 37/00

(52) U.S. Cl. 514/262.1; 514/349

(57) ABSTRACT

The invention relates to the combined administration of a PDE4 or PDE3/4 inhibitor and a disease modifying anti-rheumatic drug (DMARDs) or anti-rheumatic or anti-arthritic drug.

### FIELD OF APPLICATION OF THE INVENTION

The present invention relates to combinations of pharmaceutically active substances for use in the treatment of diseases.

The substances used in the combinations according to the invention are on the one hand known active compounds from the PDE4 and PDE3/4 inhibitors class and on the other hand so-called DMARDs (Disease Modifying Anti-Rheumatic Drugs) or related drugs.

### KNOWN TECHNICAL BACKGROUND

Rheumatoid arthritis is a chronic inflammatory disease with varying course. Despite the best therapeutic efforts, in a great number of cases the disease shows a resistant course with progressive joint destruction and physical disability.

Among the drugs used to treat rheumatoid arthritis, METHOTREXATE (INN=International Proprietary Name) is increasingly regarded as the agent of first choice because of its early onset of action and superior efficacy and tolerability.

Although METHOTREXATE can have toxic effects, making careful monitoring of patients necessary, most rheumatologists believe the benefits outweigh the risks. METHOTREXATE can diminish the activity of rheumatoid arthritis, but many patients have persistent disease even when treated with METHOTREXATE. When this occurs, rheumatologists usually add another DMARD.

There is a mounting evidence for the central role of tumour necrosis factor alpha in the pathogenesis of rheumatoid arthritis, and tumour necrosis factor alpha has emerged as a molecular target for treatment of rheumatoid arthritis.

The first such agent to be assessed in rheumatoid arthritis was INFLIXIMAB (INN), a chimeric human-murine monoclonal antibody, a specific inhibitor of tumour necrosis factor alpha. In a recent trial in a small number of patients, INFLIXIMAB in combination with a fixed low-dose (7.5 mg per week) of METHOTREXATE in rheumatoid arthritis patients with active disease despite METHOTREXATE treatment, showed enhanced degree and duration of efficacy [Maini, R N et al., Arthritis Rheum. 1998 September; 41(9):1552-63]. The combination of METHOTREXATE and a tumour necrosis factor-receptor-IgG1 fusion protein (INN: ETANERCEPT) is also effective in rheumatoid arthritis patients unresponsive to METHOTREXATE alone [Weinblatt, M E et al., N Engl J Med. 1999 Jan. 28; 340(4):253-9].

One major disadvantage of both of these anti-tumour necrosis factor alpha substances (INFLIXIMAB and ETANERCEPT) is that they can not be administered in oral form. INFLIXIMAB is administered in form of a infusion solution all 8 weeks; ETANERCEPT is administered 2 times weekly as a subcutaneous injection.

The combined use of PDE4 or PDE3/4 inhibitors and DMARDs in the sense according to the invention for therapeutic purposes has not yet been described in the prior art.

### DESCRIPTION OF THE INVENTION

It is the object of the present invention to make available a more effective therapy for disorders which can be treated with disease modifying anti-rheumatic drugs (DMARDs) or other anti-rheumatic or anti-arthritic drugs, which fulfils the following conditions:

Good oral availability

Minor side effects

Good suitability for long-term therapy

Favourable simultaneous influence on several of the inflammatory mediators

It now has been found, surprisingly, that the combined administration of a PDE4 or a PDE3/4 inhibitor together with a disease modifying anti-rheumatic drug (DMARD) or another anti-rheumatic or anti-arthritic drug, as compared with the administration of a single active ingredient from the class of disease modifying anti-rheumatic drugs (DMARDs) or other anti-rheumatic or anti-arthritic drugs alone, delays the onset and reduces the severity of symptoms of rheumatoid arthritis in the CIA-mice model (Collagen-Induced Arthritis mice model), which model is believed to be relevant to humans. The combined use of a PDE4 or a PDE3/4 inhibitor and a disease modifying anti-rheumatic drug (DMARD) or another anti-rheumatic or anti-arthritic drug thus outstandingly fulfils the above-mentioned conditions.

The invention thus relates to the combined use of a PDE4 or a PDE3/4 inhibitor and a disease modifying anti-rheumatic drug (DMARD) or another anti-rheumatic or anti-arthritic drug in the treatment of disorders which can be treated with disease modifying anti-rheumatic drugs (DMARDs) or other anti-rheumatic or anti-arthritic drugs, in particular in the treatment of disorders of the arthritic type.

“Combined use” in context of the invention means the simultaneous, sequential or separate administration of the PDE4 or PDE3/4 inhibitor on the one hand and of the disease modifying anti-rheumatic drug (DMARD) or other anti-rheumatic or anti-arthritic drug on the other hand.

Simultaneous administration includes—aside from the simultaneous uptake of two separate dosage forms containing the PDE4 or PDE3/4 inhibitor in the one and the disease modifying anti-rheumatic drug (DMARD) or the anti-rheumatic or anti-arthritic drug in the other dosage form—pharmaceutical compositions containing both active ingredients in one single dosage form (fixed unit dose form). The invention therefore relates to pharmaceutical compositions for the effective treatment of disorders which can be treated with disease modifying anti-rheumatic drugs (DMARDs) or other anti-rheumatic or anti-arthritic drugs, in particular for the treatment of disorders of the arthritic type, containing simultaneously a PDE4 or a PDE3/4 inhibitor and a disease modifying anti-rheumatic drug (DMARD) or another anti-rheumatic or anti-arthritic drug. The pharmaceutical composition of the present invention can be prepared by mixing the first active ingredient with the second active ingredient. Therefore, the invention also relates to a process for the preparation of a pharmaceutical composition which comprises mixing a first active ingredient, which is a PDE4 inhibitor or PDE3/4 inhibitor, with a second active ingredient, which is a disease modifying anti-rheumatic drug (DMARD) or anti-rheumatic or anti-arthritic drug. Simultaneous administration also includes the oral administration of the PDE4 or PDE3/4 inhibitor during i. v. administration (e. g. by infusion) of the disease modifying anti-rheumatic drug (DMARD) or the anti-rheumatic or anti-arthritic drug.

Sequential administration in the context of the invention means the administration of the PDE4 or PDE3/4 inhibitor on the one hand and of the disease modifying anti-rheumatic drug (DMARD) or other anti-rheumatic or anti-arthritic drug on the other hand in separate dosage forms within less than 12 hours, more preferably within less than one hour, most preferably within 5 minutes or less.

Separate administration within the context of the invention means the administration of the PDE4 or PDE3/4 inhibitor on the one hand and of the disease modifying anti-rheumatic drug (DMARD) or other anti-rheumatic or anti-arthritic drug on the other hand in separate dosage forms within 12 hours or more, including administration regimen where the PDE4 or PDE3/4 inhibitor is administered e. g. once or twice daily and the disease modifying anti-rheumatic drug (DMARD) or other anti-rheumatic or anti-arthritic drug is administered e. g. every second or third day, or once a week.

Sequential and separate administration also include the oral administration of the PDE4 or PDE3/4 inhibitor and the i. v. administration (e. g. by infusion) of the disease modifying anti-rheumatic drug (DMARD) or the anti-rheumatic or anti-arthritic drug.

“Combined use” in context of the invention also includes a medicament pack comprising both the PDE4 or PDE3/4 inhibitor and the disease modifying anti-rheumatic drug (DMARD) or the anti-rheumatic or anti-arthritic drug as discrete separate dosage forms, in separate containers or e. g. in blisters containing both types of drugs in discrete solid dosage units, preferably in a form in which the dosage units which have to be taken together or which have to be taken within one day are grouped together in a manner which is convenient for the patient. Said medicament pack may contain instructions for the simultaneous, sequential or separate administration of the discrete separate dosage units, to a patient in need thereof

In a still further aspect, the present invention provides a method of effective treatment of disorders which can be treated with disease modifying anti-rheumatic drugs (DMARDs) or other anti-rheumatic or anti-arthritic drugs, in particular of effective treatment of disorders of the arthritic type which comprises administering to a subject in need thereof, an effective amount of a PDE4 or PDE3/4 inhibitor and of a disease modifying anti-rheumatic drug (DMARD) or anti-rheumatic or anti-arthritic drug. Said method includes a medicament pack containing a PDE4 or PDE3/4 inhibitor and a written description that said PDE4 or PDE3/4 inhibitor can be administered together with a disease modifying anti-rheumatic drug (DMARD) or anti-rheumatic or anti-arthritic drug for the treatment of disorders which can be treated with disease modifying anti-rheumatic drugs (DMARDs) or other anti-rheumatic or anti-arthritic drugs, in particular for the treatment of disorders of the arthritic type. Likewise, said method includes a medicament pack containing a disease modifying anti-rheumatic drug (DMARD) or anti-rheumatic or anti-arthritic drug and a written description that said disease modifying anti-rheumatic drug (DMARD) or anti-rheumatic or anti-arthritic drug can be administered together with a PDE4 or PDE3/4 inhibitor for the treatment of disorders which can be treated with disease modifying anti-rheumatic drugs (DMARDs) or other anti-rheumatic or anti-arthritic drugs, in particular for the treatment of disorders of the arthritic type.

In a still further aspect, the present invention provides a method of effective treatment of disorders which can be treated with disease modifying anti-rheumatic drugs (DMARDs) or other anti-rheumatic or anti-arthritic drugs, in particular of effective treatment of disorders of the arthritic type, which method delays the onset and reduces the severity of symptoms of the disorders, and which comprises administering to a subject in need thereof, an effective amount of a PDE4 or PDE3/4 inhibitor together with a disease modifying anti-rheumatic drug (DMARD) or anti-rheumatic or anti-arthritic drug.

By the expression “PDE4 inhibitor” is meant a selective phosphodiesterase inhibitor, which inhibits preferentially the type 4 phosphodiesterase when compared to other known types of phosphodiesterase, e.g. type 1, 2, 3, 5 etc., whereby the compound has a lower IC₅₀ (more potent) for the type 4 phosphodiesterase, such as where the IC₅₀ for PDE4 inhibition is about factor 10 lower compared to IC₅₀ for inhibition of other known type of phosphodiesterase, e.g. type 1, 2, 3, 5 etc.

Analogously, the expression “PDE3/4 inhibitor” is defined. Methods to determine the activity and selectivity of a phosphodiesterase inhibitor are known to the person skilled in the art. In this connection it may be mentioned, for example, the methods described by Thompson et al. (Adv Cycl Nucl Res 10: 69-92, 1979), Giembycz et al. (Br J Pharmacol 118: 1945-1958, 1996) and the phosphodiesterase scintillation proximity assay of Amersham Pharmacia Biotech.

As possible PDE4 or PDE3/4 inhibitors within the meaning of the present invention may be mentioned, by way of example, those PDE4 or PDE3/4 inhibitors (hereinafter referred to as “EXEMPLARY PDE4 OR PDE3/4 INHIBITORS”) which are named expressis verbis as an example, or described or claimed generically in the following patent applications and patents: DE 1545687, DE 2028869, DE 2123328, DE 2315801, DE 2402908, DE 24139 35, DE 3900233, EP 0103497, EP 0139464, EP 0158380, EP 0163965, EP 0335386, EP 0389282, EP 0393500, EP 0428302, EP 0435811, EP 0449216, EP 0459505, EP 0470805, EP 0490823, EP 0506194, EP 0510562, EP 0511865, EP 0527117, EP 0553174, EP 0557016, EP 0626939, EP 0664289, EP 0671389, EP 0685474, EP 0685475, EP 0685479, EP 0731099, EP 0736532, EP 0738715, EP 0748805, EP 0763534, EP 0816357, EP 0819688, EP 0819689, EP 0832886, EP 0834508, EP 0848000, JP 92234389, JP 94329652, JP 95010875, JP 98072415, JP 98147585, U.S. Pat. No. 5,703,098, U.S. Pat. No. 5,739,144, WO 9117991, WO 9200968, WO 9212961, WO 9307146, WO 9315044, WO 9315045, WO 9318024, WO 9319068, WO 9319720, WO 9319747, WO 9319749, WO 9319751, WO 9325517, WO 9402465, WO 9412461, WO 9420455, WO 9422852, WO 9427947, WO 9500516, WO 9501338, WO 9501980, WO 9503794, WO 9504045, WO 9504046, WO 9505386, WO 9508534, WO 9509623, WO 9509624, WO 9509627, WO 9509836, WO 9514667, WO 9514680, WO 9514681, WO 9517392, WO 9517399, WO 9519362, WO 9520578, WO 9522520, WO 9524381, WO 9527692, WO 9535281, WO 9535283, WO 9535284, WO 9600218, WO 9601825, WO 9606843, WO 9603399, WO 9611690, WO 9611917, WO 9612720, WO 9631486, WO 9631487, WO 9635683, WO 9636595, WO 9636596, WO 9636611, WO 9636625, WO 9636626, WO 9636638, WO 9638150, WO 9639408, WO 9640636, WO 9703967, WO 9704779, WO 9705105, WO 9708143, WO 9709345, WO 9712895, WO 9718208, WO 9719078, WO 9720833, WO 9722585, WO 9722586, WO 9723457, WO 9723460, WO 9723461, WO 9724117, WO 9724355, WO 9725312, WO 9728131, WO 9730999, WO 9731000, WO 9732853, WO 9735854, WO 9736905, WO 9740032, WO 9743288, WO 9744036, WO 9744322, WO 9747604, WO 9748697, WO 9804534, WO 9805327, WO 9806692, WO 9806704, WO 9807715, WO 9808828, WO 9808830, WO 9808841, WO 9808844, WO 9809946, WO 9809961, WO 9811113, WO 9814448, WO 9818796, WO 9821207, WO 9821208, WO 9821209, WO 9822453, WO 9831674, WO 9840382, WO 9845268, WO 9855481, WO 9856756, WO 9905111, WO 9905112, WO 9505113, WO 9906404, WO 9918095, WO 9931071, WO 9931090, WO 9947505, WO 9957115, WO 9957118, WO 9964414, WO 0001695, WO 0012501, WO 0042017, WO 0042018, WO 0042019, WO 0042020, WO 0042034, WO 0119818, WO 0130766, WO 0130777 and WO0151470.

Exemplary PDE inhibitors are shown in International Patent Application WO 0113953 with their formulae, which are included herewith by reference.

Those PDE4 or PDE3/4 inhibitors are to be emphasized (hereinafter referred to as “SELECTED PDE4 OR PDE3/4 INHIBITORS”) which are named expressis verbis as an example and/or claimed generically in the patent applications or patents EP 0163965, EP 0389282, EP 0393500, EP 0435811, EP 0482302, EP 0499216, EP 0506194, EP 0510562, EP 0528922, EP 0553174, EP 0731099, WO 9319749, WO 9500516, WO 9501338, WO 9600218, WO 9603399, WO 9611690, WO 9636625, WO 9636626, WO 9723457, WO 9728131, WO 9735854, WO 9740032, WO 9743288, WO 9809946, WO 9807715, WO 9808841, WO 9821207, WO 9821208, WO 9821209, WO 9822453, WO 9831674, WO 9840382, WO 9855481, WO 9905111, WO 9905112, WO 9905113, WO 9931071, WO 9931090, WO 9947505, WO 9957115, WO 9957118, WO 9964414, WO 0001695, WO 0012501, WO 0042017, WO 0042018, WO 0042019, WO 0042020, WO 0042034, WO 0119818, WO 0130766, WO 0130777 and WO0151470 and the compounds with the following Research Codes (codes given by the originator): CDC-998, D-4396, SCH-351591, IC-485 and CC-1088. Substances having good oral availability are preferred here.

Preferred PDE4 or PDE3/4 inhibitors are the following compounds, which are partly only identified by their Research Codes and which are hereinafter referred to as “PREFERRED PDE4 OR PDE3/4 INHIBITORS”: CDC-998, SH-636, D-4396, SCH-351591, IC-485, CC-1088 and 3-[3-(cyclopentyloxy)-4-methoxybenzyl]-6-(ethylamino)-8-isopropyl-3H-purine [Research Code: V-11294A], N-[9-methyl-4-oxo-1-phenyl-3,4,6,7-tetrahydropyrrolo[3,2,1-jk][1,4]benzo-diazepin-3(R)-yl]pyridine-4-carboxamide [Research Code: CI-1018], 4-(3,4-dimethoxyphenyl)thiazole-2-carboxamide oxime [Research Code: ORG-20241], 3,7-dihydro-3-(4-chlorophenyl)-1-propyl-1H-purine-2,6-dione [INN AROFYLLINE], 3-[3-(cyclopentyloxy)-4-methoxybenzylamino]-1H-pyrazole-4-methanol, (−)-cis-9-ethoxy-8-methoxy-2-methyl-1,2,3,4,4a, 10b-hexahydro-6-(4-diisopropylaminocarbonylphenyl)-benzo-[c][1,6]naphthyridine [INN: PUMAFENTRINE], N-(3,5-dichloro-4-pyridinyl)-2-[1-(4-fluorobenzyl)-5-hydroxy-1H-indol-3-yl]-2-oxoacetamide [Research Code: AWD-12-281], N-(3,5-dichloropyridin-4-yl)-2-[5-fluoro-1-(4-fluorobenzyl)-1H-indol-3-yl]-2-oxoacetamide [Research Code: AWD-12-343], 8-Amino-1,3-bis(cyclopropylmethyl)xanthine [INN: CIPAMFYLLINE], tetrahydro-5-[4-methoxy-3-[(1S,2S,4R)-2-norbornyloxy]phenyl]-2(1H)-pyrimidone [INN: ATIZORAM], β-[3-(cyclopentyloxy)-4-methoxyphenyl]-1,3-dihydro-1,3-dioxo-2H-isoindole-2-propanamide [Research Code: CDC-801], methanesulfonic acid 2-(2,4-dichlorophenylcarbonyl)-3-ureidobenzo-furan-6-yl ester [Research Code: BAY-19-8004], (Z)-5-(3,5-di-tert-butyl-4-hydroxybenzylidene)-2-imidazothiazolidin-4-one [INN: DARBUFELONE], cis-[4-cyano-4-(3-cyclopentyloxy-4-methoxyphenyl)cyclohexane-1-carboxylic acid [INN: CILOMILAST] and 3-cyclopropylmethoxy-4-difluoromethoxy-N-(3,5-dichloropyrid-4-yl)-benzamide [INN: ROFLUMILAST].

Particularly preferred PDE4 or PDE3/4 inhibitors (hereinafter referred to as “PARTICULARLY PREFERRED PDE4 OR PDE3/4 INHIBITORS”) are 3-cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a,10b-hexahydro-6-(4-diisopropylaminocarbonylphenyl)-benzo-[c][1,6]naphthyridine [INN: PUMAFENTRINE].

By the expression “disease modifying anti-rheumatic drugs (DMARDs) and other anti-rheumatic and anti-arthritic drugs” those compounds are meant which are useful in the treatment of rheumatoid arthritis. Among these, the following compounds (hereinafter referred to as “DMARD COMPOUNDS”) shall be mentioned, partly also with their INNs: N-[4-[2-(2,4-diamino-6-pteridyl)ethyl]benzoyl] L glutamic acid (10-DEAZAAMINOPTERIN), 2-[3-(diethylamino)propyl]-8,8-dipropyl-2-azaspiro[4,5]decane (ATIPRI-MOD), S-triethylphosphine gold 2,3,4,6-tetra-O-acetyl-1-thio-beta-D-glucopyranoside (AURANOFIN), 6-(1-methyl-4-nitroimidazol-5-ylthio)purin (AZATHIOPRINE), 2-mercapto-2-methylpropanoyl-L-cysteine (BUCILLAMINE), 4-amino-1-beta-D-ribofuranosyl-1H-imidazo[4,5-c]pyridine-3-deazaadenosine, 7-chloro-4-(4-diethylamino-1-methylbutylamino)-quinoline (CHLOROQUINE), 7-chloro-[4-[4-ethyl(2-hydroxyethyl)amino]-1-methylbutylamino]-quinoline (HYDROXYCHLOROQUINE), 2-[8-chloro-2-(trifluoromethyl)-1,2,3,4-tetrahydroquinolin-6-yl]acetic acid, 4-acetoxy-2-(4-methylphenyl)benzothiazole, 5-methyl-N-[4-(trifluoromethyl)phenyl]-3-isoxazole-carboxamide, (1S,3R)-cis-9-(3-hydroxycyclopentyl)adenine, N-(p{[(2,4-diamino-6-pteridinyl)methyl]methylamino}benzoyl)-L-(+)-glutamic acid (METHOTREXATE), 5-hydroxy-1beta-D-ribofuranosylimidazole-4-carboxamide (MIZORIBINE), 2(S)-[4-(2,4-diaminopteridin-6-ylmethyl)-3,4-dihydro-2H-1,4-benzothiazin-7-ylcarboxamido] adipic acid, 2-[[2-(p-chlorophenyl)-4-methyl-5-oxazolyl]methoxy]-2-methylpropionic acid, (E)-5-(3,5-di-tert-butyl-4-hydroxybenzylidene)-2-ethyl-isothiazolidine-1,1-dioxide, 5-[[p-[(6-methoxy-3-pyridazinyl)sulfamoyl]phenyl]-azosalicylic acid, 2-[8-[2-[6-(methylamino)pyridyl-2-ylethoxy]-3-oxo-2-(2,2,2-trifluoroethyl)-2,3,4,5-tetrahydro-1H-2-benzazepin-4-(S)-yl]acetic acid, N-[3-[(R)-1-(2-fluoro-biphenyl-4-yl)-ethyl]-isoxazol-5-yl]-morpholine-4-carboxamidine, 2-hydroxy-5-[[4-[(2-pyridinylamino)sulfonyl]phenyl]-azo]benzoic acid (SULFASALAZINE), 3-(formylamino)-7-(methylsulfonamido)-6-phenoxy-4H-1-benzopyran-4-one, cis-2-(4-chlorophenyl)-4,5-diphenyl-4,5-dihydro-1H-imidazole, N-(4-trifluoromethylphenyl)-2-cyano-3-hydroxy-crotonamide, N-[1-[4-[4-(2-pyrimidinyl)-1-piperazinylmethyl]-phenyl]cyclopropyl]-acetamide, N2-L-methionylinterleukin 1 receptor antagonist (human isoform x reduced) (ANAKINRA), 7alpha-chloro-11beta,17alpha,21-trihydroxy-16alpha-methyl-1,4-pregnadien-3,20-dione (ALCLOMETASONE), 9-fluoro-11beta,16alpha,17,21-tetrahydroxy-pregna-1,4-dien-3,20-dione-16,17-cyclopentanonacetal-21-acetate (AMCINONIDE), 9-fluoro-11beta,17,21-trihydroxy-16beta-methylpregna-1,4-diene-3,20-dione (BETAMETHASONE), (11beta,16alpha)-16,17-[butylidenebis(oxy)]-11,21-dihydroxypregna-1,4-diene-3,20-dione (BUDESONIDE), (11beta,16beta)-21-[[[4-[(acetylamino)methyl]cyclohexyl]carbonyl]oxy-9-chloro-11,17-dihydroxy-16-methylpregna-1,4-diene-3,20-dione (CICLOMETASONE), 16alpha,17-dimethylmethylendioxy-6alpha,9-difluoro-11beta-hydroxy-1,4-pregnadien-3,20-dion-21-yl-cyclopropancarboxylate (CIPROCINONIDE), 17,21-dihydroxy-4-pregnen-3,11,20-trione (CORTISONE), (11beta,16beta)-21-(acetyloxy)-11-hydroxy-2′-methyl-5′H-pregna-1,4-dieno[17,16d]oxazole-3,20-dione (DEFLAZACORT), 9-fluoro-11beta,21-dihydroxy-16alpha-methyl-1,4-pregnadien-3,20-dione (DESOXIMETASONE), 9alpha-fluoro-16alpha-methyl-11beta,17,21-trihydroxypregna-1,4-diene-3,20-dione (DEXAMETHASONE), 6alpha,9-difluoro-11beta,17,21-trihydroxypregna-1,4-diene-3,20-dione-21-acetate-17-butyrate (DIFLUPREDNATE), (11beta)-9-fluoro-11,17,21-trihydroxypregn-4-ene-3,20-dione (FLUDROCORTISONE), 6alpha-fluoro-11beta,21-dihydroxy-16alpha,17-isopropylidenedioxy-pregn-4-ene-3,20-dione (FLUDROXYCORTIDE), 6alpha-fluoro-11beta,21-dihydroxy-16alpha,17-isopropylidenedioxy-pregna-1,4-diene-3,20-dione (FLUNISOLIDE), (6alpha,11beta,16alpha)-6,9-difluoro-11,21-dihydroxy-16,17-[(1-methylethylidene)bis(oxy)]pregna-1,4-diene-3,20-dione (FLUOCINOLONE ACETONIDE), (6alpha,11beta,16alpha,)-21-(acetyloxy)-6,9-difluoro-11-hydroxy-16,17-[(1-methylethylidene)bis[oxy]-pregna-1,4-diene-3,20-dione (FLUOCINOLONE), 6alpha-fluoro-11beta-hydroxy-16alpha-methyl-3,20-dioxopregna-1,4-dien-21-oic acid (FLUOCORTIN), (6alpha,11beta,16alpha)-6-fluoro-11,21-dihydroxy-16-methylpregna-1,4-diene-3,20-dione (FLUOCORTOLONE), (6alpha,11beta)-9-fluoro-11,17-dihydroxy-6-methyl-pregna-1,4-diene-3,20-dione (FLUOROMETHOLONE), 9-fluoro-11beta,17,21-trihydroxy-16-methylene-pregna-1,4-diene-3,20-dione (FLUPREDNIDENE), (11beta, 16alpha,)-21-(acetyloxy)-3-(2-chloroethoxy)-9-fluoro-11-hydroxy-16,17-[(1-methylethylidenebis(oxy)]-20-oxopregna-3,5-diene-6-carboxaldehyde (FORMOCORTAL), 21-chloro-9-fluoro-11beta-hydroxy-16alpha,17-isopropylidenedioxy-pregn-4-ene-3,20-dione (HALCINONIDE), 2-chloro-6alpha,9-difluoro-11beta,17,21-trihydroxy-16alpha-methyl-1,4-pregnadien-3,20-dione (HALOMETASONE), 11beta-hydroxy-6alpha-methyl-4-pregnen-3,20-dione (MEDRYSONE), 11beta,17alpha,21-trihydroxy-6alpha-methylpregna-1,4-diene-3,20-dione (METHYLPREDNISOLONE), 9,21-dichloro-11beta,17-dihydroxy-16alpha-methylpregna-1,4-diene-3,20-dion 17-furoate (MOMETASONE FUROATE), 6alpha-fluoro-11beta,17,21-trihydroxy-16alpha-methylpregna-1,4-diene-3,20-dione (PARAMETHASONE), 6alpha-fluoro-11beta,17,21-trihydroxy-16alpha-methylpregna-1,4-diene-3,20-dione-21-acetate (PARAMETHASONE ACETATE), 11beta,17,21-trihydroxypregna-1,4-diene-3,20-dione 21-diethylamino-acetate (PREDNISOLAMATE), 11beta,17alpha,21-trihydroxypregna-1,4-diene-3,20-dione (PREDNISOLONE), 1,4-pregnadiene-17alpha,21-diol-3,11,10-trione (PREDNISONE), 16-methylene-11beta,17alpha,21-trihydroxypregna-1,4-diene-3,20-dione (PREDNYLIDENE), 17beta-methoxy-3-propoxyestra-1,3,5,(10)-triene (PROMESTRIENE), [(1,2-dicarboxyethyl)thio]gold disodium salt (SODIUM AUROTHIOMALATE), alpha-amino-beta-methyl-beta-mercatobutyric acid (PENICILLAMINE), [r-[R*,R*-(E)]]-cyclic-(L-alanyl-D-alanyl-N-methyl-L-leucyl N-methyl-L-leucyl-N-methyl-L-valyl-3-hydroxy-N,4-dimethyl-L-2-amino-6-ocentyl-L-2-aminobutyryl-N-methylglycyl-N-methyl L-leucyl-L-valyl-N-methyl-L-leucyl (CYCLOSPORIN) and 2-[bis(2-chloroethyl)amino]tetrahydro-2H-1,3,2-oxazophosphorine-2-oxide monohydrate (CYCLOPHOSPHAMIDE).

As selected disease modifying anti-rheumatic drugs (DMARDs) and anti-rheumatic and anti-arthritic drugs (hereinafter referred to as “SELECTED DMARD COMPOUNDS”) the following compounds shall be named by way of example: S-triethylphosphine gold 2,3,4,6-tetra-O-acetyl-1-thio-beta-D-glucopyranoside (AURANOFIN), 6-(1-methyl-4-nitroimidazol-5-ylthio)purin (AZATHIOPRINE), 7-chloro-4-(4-diethylamino-1-methylbutylamino)-quinoline (CHLOROQUINE), 7-chloro-4-[4-[ethyl(2-hydroxyethyl)amino]-1-methylbutylamino]-quinoline (HYDROXYCHLOROQUINE), 5-methyl-N-[4-(trifluoromethyl)phenyl]-3-isoxazole-carboxamide, N-(p{[(2,4-diamino-6-pteridinyl)methyl]methylamino}-benzoyl)-L-(+)-glutamic acid (METHOTREXATE), 2-hydroxy-5-[[4-[(2-pyridinylamino)sulfonyl]phenyl]-azo]benzoic acid (SULFASALAZINE), N2-L-methionylinterleukin 1 receptor antagonist (human isoform x reduced) (ANAKINRA), 7alpha-chloro-11beta,17alpha,21-trihydroxy-16alpha-methyl-1,4-pregnadien-3,20-dione (ALCLOMETASONE), 9-fluoro-11beta,16alpha,17,21-tetrahydroxy-pregna-1,4-dien-3,20-dione-16,17-cyclopentanonacetal-21-acetate (AMCINONIDE), 9-fluoro-11beta,17,21-trihydroxy-16beta-methylpregna-1,4-diene-3,20-dione (3(BETAMETHASONE), (11beta,16alpha)-16,17-[butylidenebis(oxy)]-11,21-dihydroxypregna-1,4-diene-3,20-dione (BUDESONIDE), (11beta,16beta)-21-[[[4-[(acetylamino)methyl]cyclohexyl]carbonyl]oxy-9-chloro-11,17-dihydroxy-16-methylpregna-1,4-diene-3,20-dione (CICLOMETASONE), 16alpha,17-dimethylmethylendioxy-6alpha,9-difluoro-11beta-hydroxy-1,4-pregnadien-3,20-dion-21-yl-cyclopropancarboxylate (CIPROCINONIDE), 17,21-dihydroxy-4-pregnen-3,11,20-trione (CORTISONE), (11beta,16beta)-21-(acetyloxy)-11-hydroxy-2′-methyl-5′H-pregna-1,4-dieno[17,16d]oxazole-3,20-dione (DEFLAZACORT), 9-fluoro-11beta,21-dihydroxy-16alpha-methyl-1,4-pregnadien-3,20-dione (DESOXIMETASONE), 9alpha-fluoro-16alpha-methyl-11beta,17,21-trihydroxypregna-1,4-diene-3,20-dione (DEXAMETHASONE), 6alpha,9-difluoro-11beta,17,21-trihydroxypregna-1,4-diene-3,20-dione-21-acetate-17-butyrate (DIFLUPREDNATE), (11beta)-9-fluoro-11,17,21-trihydroxypregn-4-ene-3,20-dione (FLUDROCORTISONE), 6alpha-fluoro-11beta,21-dihydroxy-16alpha,17-isopropylidenedioxy-pregn-4-ene-3,20-dione (FLUDROXYCORTIDE), 6alpha-fluoro-11beta,21-dihydroxy-16alpha,17-isopropylidenedioxy-pregna-1,4-diene-3,20-dione (FLUNISOLIDE), (6alpha,11beta,16alpha)-6,9-difluoro-11,21-dihydroxy-16,17-[(1-methyl-ethylidene)bis(oxy)]pregna-1,4-diene-3,20-dione (FLUOCINOLONE ACETONIDE), (6alpha,11beta,16alpha,)-21-(acetyloxy)-6,9-difluoro-11-hydroxy-16,17-[(1-methylethylidene)bis[oxy]-pregna-1,4-diene-3,20-dione (FLUOCINONIDE), 6alpha-fluoro-11beta-hydroxy-16alpha-methyl-3,20-dioxopregna-1,4-dien-21-oic acid (FLUOCORTIN), (6alpha,11beta,16alpha)-6-fluoro-11,21-dihydroxy-16-methylpregna-1,4-diene-3,20-dione (FLUOCORTOLONE), (6alpha,11beta)-9-fluoro-11,17-dihydroxy-6-methyl-pregna-1,4-diene-3,20-dione (FLUOROMETHOLONE), 9-fluoro-11beta,17,21-trihydroxy-16-methylene-pregna-1,4-diene-3,20-dione (FLUPREDNIDENE), (11beta, 16alpha,)-21-(acetyloxy)-3-(2-chloroethoxy)-9-fluoro-11-hydroxy-16,17-[(1-methylethylidenebis(oxy)]-20-oxopregna-3,5-diene-6-carboxaldehyde (FORMOCORTAL), 21-chloro-9-fluoro-11beta-hydroxy-16alpha,17-isopropylidenedioxy-pregn-4-ene-3,20-dione (HALCINONIDE), 2-chloro-6alpha,9-difluoro-11beta,17,21-trihydroxy-16alpha-methyl-1,4-pregnadien-3,20-dione (HALOMETASONE), 11beta-hydroxy-6alpha-methyl-4-pregnen-3,20-dione (MEDRYSONE), 11beta,17alpha,21-trihydroxy-6alpha-methylpregna-1,4-diene-3,20-dione (METHYLPREDNISOLONE), 9,21-dichloro-11beta,17-dihydroxy-16alpha-methylpregna-1,4-diene-3,20-dion 17-furoate (MOMETASONE FUROATE), 6alpha-fluoro-11beta,17,21-trihydroxy-16alpha-methylpregna-1,4-diene-3,20-dione (PARAMETHASONE), 6alpha-fluoro-11beta,17,21-trihydroxy-16alpha-methylpregna-1,4-diene-3,20-dione-21-acetate (PARAMETHASONE ACETATE), 11beta,17,21-trihydroxypregna-1,4-diene-3,20-dione 21-diethylamino-acetate (PREDNISOLAMATE), 11beta,17alpha,21-trihydroxypregna-1,4-diene-3,20-dione (PREDNISOLONE), 1,4-pregnadiene-17alpha,21-diol-3,11,10-trione (PREDNISONE), 16-methylene-11beta,17alpha,21-trihydroxypregna-1,4-diene-3,20-dione (PREDNYLIDENE), 17beta-methoxy-3-propoxyestra-1,3,5,(10)-triene (PROMESTRIENE), [(1,2-dicarboxyethyl)thio]gold disodium salt (SODIUM AUROTHIOMALATE), alpha-amino-beta-methyl-beta-mercatobutyric acid (PENICILLAMINE), [r-[R*,R*-(E)]]-cyclic-(L-alanyl-D-alanyl-N-methyl-L-leucyl N-methyl-L-leucyl-N-methyl-L-valyl-3-hydroxy-N,4-dimethyl-L-2-amino-6-ocentyl-L-2-aminobutyryl-N-methylglycyl-N-methyl L-leucyl-L-valyl-N-methyl-L-leucyl (CYCLOSPORIN) and 2-[bis(2-chloroethyl)amino]tetrahydro-2H-1,3,2-oxazophosphorine-2-oxide monohydrate (CYCLOPHOSPHAMIDE).

As preferred disease modifying anti-rheumatic drugs (DMARDs) and anti-rheumatic and anti-arthritic drugs (hereinafter referred to as “PREFERRED DMARD COMPOUNDS”) the following compounds shall be named: S-triethylphosphine gold 2,3,4,6-tetra-O-acetyl-1-thio-beta-D-glucopyranoside (AURANOFIN), 6-(1-methyl-4-nitroimidazol-5-ylthio)purin (AZATHIOPRINE), 7-chloro-4-(4-diethylamino-1-methylbutylamino)-quinoline (CHLOROQUINE), 7-chloro-4-[4-[ethyl(2-hydroxyethyl)amino]-1-methylbutylamino]-quinoline (HYDROXYCHLOROQUINE), 5-methyl-N-[4-(trifluoromethyl)phenyl]-3-isoxazole-carboxamide, N-(p{[(2,4-diamino-6-pteridinyl)methyl]methylamino}benzoyl)-L-(+)-glutamic acid (METHOTREXATE), 2-hydroxy-5-[[4-[(2-pyridinylamino)sulfonyl]phenyl]-azo]benzoic acid (SULFASALAZINE), N2-L-methionylinterleukin 1 receptor antagonist (human isoform x reduced) (ANAKINRA), 17,21-dihydroxy-4-pregnen-3,11,20-trione (CORTISONE), [(1,2-dicarboxyethyl)thio]gold disodium salt (SODIUM AUROTHIOMALATE), alpha-amino-beta-methyl-beta-mercatobutyric acid (PENICILLAMINE), [r-[R*,R*-(E)]]-cyclic-(L-alanyl-D-alanyl-N-methyl-L-leucyl N-methyl-L-leucyl-N-methyl-L-valyl-3-hydroxy-N,4-dimethyl-L-2-amino-6-ocentyl-L-2-aminobutyryl-N-methylglycyl-N-methyl L-leucyl-L-valyl-N-methyl-L-leucyl (CYCLOSPORIN) and 2-[bis(2-chloroethyl)amino]tetrahydro-2H-1,3,2-oxazophosphorine-2-oxide monohydrate (CYCLOPHOSPHAMIDE).

As particularly preferred disease modifying anti-rheumatic drugs (DMARDs) and anti-rheumatic and anti-arthritic drugs (hereinafter referred to as “PARTICULARLY PREFERRED DMARD COMPOUNDS”) the following compounds shall be named: 5-methyl-N-[4-(trifluoromethyl)phenyl]-3-isoxazole-carboxamide, N-(p{[(2,4-diamino-6-pteridinyl)methyl]methylamino}benzoyl)-L-(+)-glutamic acid (METHOTREXATE) and N2-L-methionylinterleukin 1 receptor antagonist (human isoform x reduced) (ANAKINRA).

In the context of the present invention, unless otherwise stated, the compounds named expressly can be used as such or in the form of their pharmacologically acceptable derivatives. A pharmacologically acceptable derivative means in particular a pharmacologically acceptable salt or solvate (e. g. hydrate), a pharmacologically acceptable solvate of such salt, a pharmacologically acceptable N-oxide or a pharmacologically acceptable salt or solvate of the latter.

Suitable pharmacologically acceptable salts here are on the one hand in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation—depending on whether it is a mono- or polybasic acid and depending on which salt is desired—in an equimolar quantitative ratio or one differing therefrom. Furthermore, the active compounds mentioned can also be present as pure enantiomers or as enantiomer mixtures in any mixing ratio.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Certain of the active ingredients used in the present invention are capable of existing in stereoisomeric forms. The invention encompasses all stereoisomers of the active ingredients and mixtures thereof including racemates. Tautomers and mixtures thereof of the active ingredients are also part of the present invention.

In the context of the present invention, a pharmaceutically acceptable derivative of METHOTREXATE means a pharmaceutically acceptable salt or solvate (e. g. hydrate) or a pharmaceutically acceptable solvate of such salt. Particularly preferred in this connection is the disodium salt of METHOTREXATE.

Disorders which can be treated with disease modifying anti-rheumatic drugs (DMARDs) or other anti-rheumatic or anti-arthritic drugs are summarised below. Disorders of the arthritic type which may be mentioned in particular, are rheumatoid arthritis, rheumatoid spondylitis and osteoarthritis.

One embodiment of the invention is the combined use of an EXEMPLARY PDE4 OR PDE3/4 INHIBITOR and a DMARD COMPOUND in the treatment of the above-mentioned disorders.

A further embodiment of the invention is the combined use of a SELECTED PDE4 OR PDE3/4 INHIBITOR and a SELECTED DMARD COMPOUND in the treatment of the above-mentioned disorders.

A preferred embodiment of the invention is the combined use of a PREFERRED PDE4 OR PDE3/4 INHIBITOR and a PREFERRED DMARD COMPOUND in the treatment of the above-mentioned disorders.

A second preferred embodiment of the invention is the combined use of PARTICULARLY PREFERRED PDE4 OR PDE3/4 INHIBITOR and a DMARD COMPOUND in the treatment of the above-mentioned disorders.

A third preferred embodiment of the invention is the combined use of a PARTICULARLY PREFERRED PDE4 OR PDE3/4 INHIBITOR and a SELECTED DMARD COMPOUND in the treatment of the above-mentioned disorders.

A fourth preferred embodiment of the invention is the combined use of a PARTICULARLY PREFERRED PDE4 OR PDE3/4 INHIBITORS and a PREFERRED DMARD COMPOUND in the treatment of the above-mentioned disorders.

A particularly preferred embodiment of the invention is the combined use of a PARTICULARLY PREFERRED PDE4 OR PDE3/4 INHIBITOR and a PARTICULARLY PREFERRED DMARD COMPOUND in the treatment of the above-mentioned disorders.

Within the embodiments, preferred embodiments and particularly preferred embodiments those have to be mentioned particularly where the PDE4 or PDE3/4 inhibitor is administered orally.

Commercial Utility

On account of their properties, the combinations of PDE4 or PDE3/4 inhibitors and disease modifying anti-rheumatic drugs (DMARDs) or other anti-rheumatic or anti-arthritic drugs according to the invention can be employed in human and veterinary medicine and therapeutics, where they can be used, for example, for the treatment and prophylaxis of the following illnesses: acute and chronic (in particular inflammatory and allergen-induced) airway disorders of various origins (bronchitis, allergic bronchitis, bronchial asthma, emphysema, COPD); dermatoses (especially of proliferative, inflammatory and allergic type) such as, for example, psoriasis (vulgaris), toxic and allergic contact eczema, atopic eczema, seborrheic eczema, lichen simplex, sunburn, pruritus in the anogenital area, alopecia areata, hypertrophic scars, discoid lupus erythematosus, follicular and wide-area pyodermias, endogenous and exogenous acne, acne rosacea and other proliferative, inflammatory and allergic skin disorders; disorders which are based on an excessive release of TNF and leukotrienes, in particular disorders of the arthritis type (rheumatoid arthritis, rheumatoid spondylitis, osteoarthritis and other arthritic conditions, such as psoriatic and juvenile arthritis), disorders of the immune system (AIDS, multiple sclerosis), graft-versus-host reactions, transplant rejection reactions, symptoms of shock [septic shock, endotoxin shock, gram-negative sepsis, toxic shock syndrome and ARDS (adult respiratory distress syndrome)], and generalized inflammations in the gastrointestinal area (Crohn's disease and ulcerative colitis); disorders which are based on allergic and/or chronic, faulty immunological reactions in the area of the upper airways (pharynx, nose) and the adjacent regions (paranasal sinuses, eyes), such as, for example, chronic rhinitis/sinusitis.

Medicaments which contain the PDE4 or PDE3/4 inhibitor and the disease modifying anti-rheumatic drug (DMARD) or other anti-rheumatic or anti-arthritic drug, either alone or in a fixed combination, are prepared by processes which are known per se and familiar to the person skilled in the art. As medicaments, the pharmacologically active compounds (=active compounds) are employed either as such, or preferably in combination with suitable pharmaceutical excipients or vehicles in the form of tablets, coated tablets, capsules, suppositories, patches (e.g. as TTS), emulsions, suspensions or solutions, the active compound content advantageously being between 0.1 and 95% and it being possible, by the appropriate choice of the excipients and vehicles, to obtain a pharmaceutical administration form exactly suited to the active compound and/or to the desired onset of action and/or to the duration of action (e.g. a delayed-release form or an enteric form). The preferred administration form in the context of the invention, at least for the PDE4 or a PDE3/4 inhibitor, is the oral administration in the form of tablets, coated tablets, capsules and the like. The particularly preferred administration form in the context of the invention is the oral administration for both the PDE4 or a PDE3/4 inhibitor and the disease modifying anti-rheumatic drug (DMARD) or other anti-rheumatic or anti-arthritic drug.

The person skilled in the art is familiar on the basis of his/her expert knowledge with excipients or vehicles which are suitable for the desired pharmaceutical formulations. In addition to solvents, gel formers, suppository bases, tablet excipients and other active compound carriers, it is possible to use, for example, antioxidants, dispersants, emulsifiers, antifoams, flavour corrigents, preservatives, solubilizers, colorants or in particular permeation promoters and complexing agents (e.g. cyclodextrins).

As outlined above, “combined use”, “combined administration” and “combination” in the sense of the present invention is to be understood as meaning that the individual components [i.e. the PDE4 or a PDE3/4 inhibitor on the one hand and the disease modifying anti-rheumatic drug (DMARD) or other anti-rheumatic or anti-arthritic drug on the other hand] can be administered in a manner which is known and customary per se simultaneously (in the form of a combination medicament), more or less at the same time (from separate pack units) or successively (directly one after the other or else with a relatively large time interval).

In the case of administration of the individual components more or less at the same time from separate pack units and in the case of the administration of the individual components which takes place one after the other, it is possible, if desired, to select a different administration form. For example, one component can be administered orally, while the other component is administered intravenously.

For the above-mentioned therapeutic uses, the dosages administered will, of course, vary with the first and second active ingredients employed, the mode of administration, the treatment desired and the disorder indicated. In general, the active ingredients are administered in the dose customary for them.

As an example, satisfactory results will be obtained when the total daily dosage of the first active ingredient(s), the PDE4 respectively the PDE3/4 inhibitor, when taken orally is in the range from 1-2000 μg/kg of body weight. In the case of the particularly preferred PDE4 inhibitor ROFLUMILAST, the daily dosage is in a range from 1-20 μg/kg of body weight. The daily dosage for the particularly preferred PDE3/4 inhibitor PUMAFENTRINE is in a range from 300-1500 μg/kg of body weight.

### Pharmacology

Summary:

Collagen-induced arthritis (CIA) in DBA/1 mice: Mice were immunized intradermally with 200 μg/mouse of type II (CII) bovine collagen in Freund's complete adjuvant (FCA) and challenged intraperitoneally 21 days later with 200 μg/mouse of CII in saline. The mice were then administered orally either with 1 mg/kg/day of 3-Cyclopropylmethoxy-4-difluoromethoxy-N-(3,5-dichloropyrid-4-yl)-benzamide [INN: ROFLUMILAST], 3 mg/kg/day of (−)-cis-9-ethoxy-8-methoxy-2-methyl-1,2,3,4,4a,10b-hexahydro-6-(4-diisopropylaminocarbonylphenyl)-benzo[c][1,6]naphthyridine hydrochloride [INN: PUMAFENTRINE hydrochloride], 1 mg/kg/day of METHOTREXATE, or with combinations of METHOTREXATE and the PDE inhibitors for 10 consecutive days, starting 2 days after challenge injection. Control mice received either METHOTREXATE alone (1 mg/kg/day) or the vehicle only.

3-Cyclopropylmethoxy-4-difluoromethoxy-N-(3,5-dichloropyrid-4-yl)-benzamide [INN: ROFLUMILAST] and (−)-cis-9-ethoxy-8-methoxy-2-methyl-1,2,3,4,4a,10b-hexahydro-6-(4-diisopropylaminocarbonyl-phenyl)-benzo-[c][1,6]naphthyridine hydrochloride [INN: PUMAFENTRINE hydrochloride] were found to be effective in reducing the severity of arthritis compared with control mice, with both compounds showing similar efficacy. Furthermore, when used in combination with METHOTREXATE, a highly significant therapeutic effect was observed in both treatment groups.

In contrast, METHOTREXATE alone at the dose used failed to produce a significant clinical improvement compared with the control mice.

It is concluded that given orally, treatments with ROFLUMILAST+METHOTREXATE or PUMAFENTRINE hydrochloride+METHOTREXATE have at least additive beneficial effects in delaying the onset and reducing the severity of CIA in DBA/1 mice.

Experimental Protocol:

The experimental protocol is schematically represented in FIG. 1.

DBA/1 mice were obtained from Harlan Olac (Bicester, UK) and used 8 weeks old. CIA was induced as described previously (Ruchatz, H., et al., J Immunol. 1998, 160(11):5654-60). Briefly, mice were immunised by intradermal injection of 200 μg of acidified type II collagen (Sigma, Poole UK) emulsified in Freund's Complete adjuvant (FCA, Difco, Detroit, Mich., USA). Collagen (200 μg in PBS) was injected intraperitoneally on day 21 (challenge). Mice were individually ear marked for identification. They were monitored daily and scored every other day for signs of arthritis as follows: 0=normal, 1=erythema, 2=erythema plus swelling, 3=extension/loss of function, and total score=sum of four limbs. Paw thickness was measured with a dial-calliper (Kroeplin, Munich, Germany). Compounds 3-Cyclopropylmethoxy-4-difluoromethoxy-N-(3,5-dichloropyrid-4-yl)-benzamide [INN: ROFLUMILAST] (1 mg/kg), (−)-cis-9-ethoxy-8-methoxy-2-methyl-1,2,3,4,4a,10b-hexahydro-6-(4-diisopropylaminocarbonylphenyl)-benzo-[c][1,6]naphthyridine hydrochloride [INN: PUMAFENTRINE hydrochloride] (3 mg/kg, calculated for PUMAFENTRINE), METHOTREXATE (1 mg/kg), or combinations were administered orally daily by a gavage tube for 10 consecutive days. The compound suspensions were prepared fresh daily just prior to administration. To this end, compounds were suspended (using an Ultra Turrax) in the following vehicle: 4% aqueous hydroxymethyl cellulose (Sigma) solution containing 2% polyethylenglycol 400 (PEG400, Sigma). Control mice were administered with the vehicle only. Treatment was withdrawn on day 33 and the mice monitored for a further 8 days. Influence of drug treatments on mean clinical index, arthritic paws and paw thickness were analysed by one-way ANOVA at days 31, 35, and 39. Between group differences in incidence of arthritis were analysed by chi-square test.

### RESULTS

Detailed results are presented in FIGS. 2 and 3.

Vehicle control mice developed arthritis, which was evident in a profound increase of mean paw thickness (FIGS. 2 and 3). Similarily, arthritis was apparent in terms of mean clinical scores, mean number of arthritic paws, and incidence (not shown). Disease first appeared 2-3 days after challenge injection. Mice treated with 3-Cyclopropylmethoxy-4-difluoromethoxy-N-(3,5-dichloropyrid-4-yl)benzamide [INN: ROFLUMILAST] or (−)-cis-9-ethoxy-8-methoxy-2-methyl-1,2,3,4,4a,10b-hexahydro-6-(4-diisopropylaminocarbonylphenyl)-benzo-[c][1,6]naphthyridine hydrochloride [INN: PUMAFENTRINE hydrochloride] developed significantly less severe disease on days 35 and 39 when compared with control mice, with both compounds showing similar efficacy. In addition, when used in combination with METHOTREXATE, a highly significant therapeutic effect was observed in both treatment groups on all clinical parameters scored (days 31, 35, 39; shown are paw thicknesses only in FIGS. 2 and 3). Finally, METHOTREXATE alone in the dose used (1 mg/kg) failed to demonstrate any clinical improvement when compared with control mice.

### DESCRIPTION OF THE FIGURES

FIG. 1: Experimental protocol and treatment regimen.

FIG. 2: Anti-arthritic effect of METHOTREXATE (MTX, 1 mg/kg), ROFLUMILAST (1 mg/kg), or the drug combination on paw thickness in murine CIA. **P<0.01 vs. Vehicle control; #P<0.05 vs. ROFLUMILAST alone; ##P<0.01 vs. ROFLUMILAST alone.

FIG. 3: Anti-arthritic effect of METHOTREXATE (MTX, 1 mg/kg), PUMAFENTRINE hydrochloride (Pumafentrine, 3 mg/kg), or the drug combination on paw thickness in murine CIA. **P<0.01 vs. Vehicle control; #P<0.05 vs. PUMAFENTRINE hydrochloride alone.

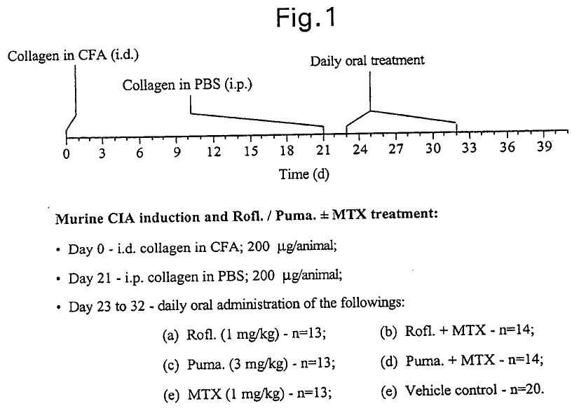

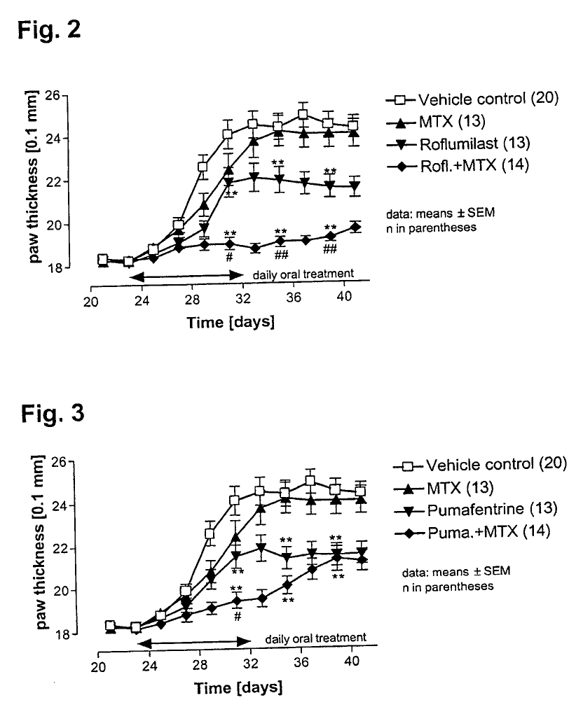

1.-13. (canceled)

14. A method of treating a disease or disorder in a patient comprising administering to a patient in need thereof a therapeutically effective amount of a pharmaceutical composition comprising a first active ingredient which is a PDE4 inhibitor, and a second active ingredient which is a disease modifying anti-rheumatic drug (DMARD) and suitable pharmaceutical excipients, wherein the PDE4 inhibitor is selected from the group consisting of 3-cyclopropylmethoxy-4-difluoromethoxy-N-(3,5-dichloropyrid-4-yl)-benzamide [INN: ROFLUMILAST] and pharmacologically acceptable salts, hydrates, solvates, hydrates of the salts, solvates of the salts, N-oxides, salts of the N-oxides, hydrates of the N-oxides and solvates of the N-oxides thereof, and wherein the disease modifying anti-rheumatic drug (DMARD) is selected from the group consisting of N-(p-{[(2,4-diamino-6-pteridinyl)methyl]methylamino}benzoyl)-L-(+)-glutamic acid (METHOTREXATE) and pharmacologically acceptable salts, hydrates, solvates, hydrates of the salts, solvates of the salts, N-oxides, salts of the N-oxides, hydrates of the N-oxides and solvates of the N-oxides thereof.

15. The method according to claim 14, wherein the PDE4 inhibitor is 3-cyclopropylmethoxy-4-difluoromethoxy-N-(3,5-dichloropyrid-4-yl)-benzamide.

16. The method according to claim 14, wherein the PDE4 inhibitor is 3-cyclopropylmethoxy-4-difluoromethoxy-N-(3,5-dichloro-1-oxy-pyrid-4-yl)-benzamide.

17. The method according to claim 14, wherein the disease modifying anti-rheumatic drug (DMARD) is N-(p-{[(2,4-diamino-6-pteridinyl)methyl]methylamino}-benzoyl)-L-(+)-glutamic acid (METHOTREXATE).

18. The method according to claim 14, wherein the disease modifying anti-rheumatic drug (DMARD) is the di-sodium salt of N-(p-{[(2,4-diamino-6-pteridinyl)methyl]methyl-amino}benzoyl)-L-(+)-glutamic acid.

19. The method according to claim 14, wherein the first active ingredient which is a PDE4 inhibitor and the second active ingredient which is a disease modifying anti-rheumatic drug (DMARD) are in one single dosage form.

20. The method according to claim 19, wherein the single dosage form is suitable for oral administration.

21. The method according to claim 14, wherein a first dosage form comprises the first active ingredient which is a PDE4 inhibitor and a second dosage form comprises the second active ingredient which is a disease modifying anti-rheumatic drug (DMARD).

22. The method according to claim 21, wherein the first dosage form is suitable for oral administration and wherein the second dosage form is suitable for intravenous administration.

23. The method according to claim 21, wherein the first dosage form and the second dosage form are suitable for oral administration.

24. The method according to claim 14, wherein the disease or disorder is selected from the group consisting of acute airway disorders, chronic airway disorders, dermatoses, disorders of the arthritis type, disorders of the immune system, graft-versus-host reactions, transplant rejection reactions, symptoms of shock, generalized inflammations in the gastrointestinal area, and disorders which are based on allergic or chronic faulty immunological reactions in the area of the upper airways.

25. The method according to claim 14, wherein the disease or disorder is selected from the group consisting of bronchitis, allergic bronchitis, bronchial asthma, emphysema, COPD, psoriasis, toxic eczema, allergic contact eczema, atopic eczema, seborrheic eczema, lichen simplex, sunburn, pruritis, alopecia areata, hypertrophic scars, discoid lupus erythematosus, follicular and wide-area pyodermias, endogenous acne, exogenous acne, acne rosacea, rheumatoid arthritis, rheumatoid spondylitis, osteoarthritis, psoriatic arthritis, juvenile arthritis, AIDS, multiple sclerosis, septic shock, endotoxin shock, gram-negative sepsis, toxic shock syndrome, ARDS, Crohn's disease, ulcerative colitis, allergic rhinitis and allergic sinusitis.
